(19) United States
(12) Patent Application Publication
(10) Pub. No.: US 2026/0130574 A1
(43) Pub. Date: May 14, 2026

(54) ENDOSCOPIC DIAGNOSIS SUPPORT DEVICE, ENDOSCOPIC DIAGNOSIS SUPPORT METHOD, AND ENDOSCOPIC DIAGNOSIS SUPPORT PROGRAM

(71) Applicant: OLYMPUS MEDICAL SYSTEMS CORP., Tokyo (JP)

(72) Inventors: Osamu NONAKA, Machida-shi (JP); Akihiro KUBOTA, Kokubunji-shi (JP)

(73) Assignee: OLYMPUS MEDICAL SYSTEMS CORP., Tokyo (JP)

(21) Appl. No.: 19/413,535

(22) Filed: Dec. 9, 2025

Related U.S. Application Data
(63) Continuation of application No. PCT/JP2023/034963, filed on Sep. 26, 2023.

(51) Int. Cl. A61B 1/00; A61B 1/31; G06T 7/00

(52) U.S. Cl. CPC A61B 1/000096; A61B 1/00006; A61B 1/00045; A61B 1/31; G06T 7/0012; G06T 2207/10068; G06T 2207/20081; G06T 2207/20084; G06T 2207/30028; G06T 2207/30096

(57) ABSTRACT

An endoscopic diagnosis support device includes: an area determination portion configured to specify a current passage area of a lumen, which has been imaged in an image of the lumen of a hollow organ; a feature determination portion configured to determine features of the current passage area; and a diagnosis support information generation portion configured to generate support information for accessing or diagnosing a planned passage area in the lumen based on features of the current passage area.

### CROSS-REFERENCE TO RELATED APPLICATIONS

This application is a continuation application based on PCT Patent Application No. PCT/JP2023/034963, filed on Sep. 26, 2023, the entire content of which is hereby incorporated by reference.

### FIELD

The present disclosure relates to an endoscopic diagnosis support device, an endoscopic diagnosis support method, and an endoscopic diagnosis support program.

### BACKGROUND

In the related art, computer aided detection/diagnosis (CAD), which analyzes endoscopic images by a computer and supports a surgeon in diagnosis, has been developed. In diagnosis in a lumen of a hollow organ by an endoscope, there are individual differences for each patient, and thus it is desirable to provide diagnosis support that takes into account the individual differences in the lumen for each patient. It is particularly desirable to provide suitable diagnosis support to a surgeon who has little diagnosis experience. The individual differences in the lumen for each patient should be taken into account not only in the diagnosis support, but also in a process of accessing a diagnosis part leading up to the diagnosis, as well as in screening and treatment. Hereafter, support for medical professionals under these circumstances will be collectively referred to as diagnosis support.

Japanese Unexamined Patent Application, First Publication No. 2021-153808 (Patent Document 1) describes a diagnosis support system that provides information related to the operation of an endoscope to perform support diagnosis. The diagnosis support system described in Patent Document 1 acquires a three-dimensional medical image of the inside of the body using a means capable of capturing a three-dimensional image of the inside of the body, such as an X-ray CT, an X-ray cone beam CT, an MRI-CT, or ultrasound diagnostic device, generates a virtual endoscopic image reconstructed from the acquired three-dimensional medical image, and outputs operational support information regarding the operation of the endoscope.

However, the diagnosis support system described in Patent Document 1 requires that a virtual endoscopic image be acquired in advance before surgery. In particular, when an emergency treatment is required for a patient, it is difficult to acquire a virtual endoscopic image in advance before the surgery.

### SUMMARY

The present disclosure provides an endoscopic diagnosis support device, an endoscopic diagnosis support method, and an endoscopic diagnosis support program that can provide diagnosis support that takes into account individual differences in a lumen of a hollow organ for each patient without requiring any preliminary work before surgery.

According to a first aspect of the present disclosure, there is provided an endoscopic diagnosis support device including: an area determination portion configured to specify a current passage area of a lumen, which has been imaged in an image of the lumen of a hollow organ; a feature determination portion configured to determine features of the current passage area; and a diagnosis support information generation portion configured to generate support information for accessing or diagnosing a planned passage area in the lumen based on features of the current passage area.

The endoscopic diagnosis support device, the endoscopic diagnosis support method, and the endoscopic diagnosis support program of the present disclosure can provide diagnosis support that takes into account individual differences in a lumen of a hollow organ for each patient without requiring any preliminary work before the surgery.

### BRIEF DESCRIPTION OF THE DRAWINGS

FIG. 1 is a view showing an endoscope system according to one embodiment.

FIG. 2 is a functional block diagram of the endoscope system.

FIG. 3 is a view illustrating diagnosis support information.

FIG. 4 is a view showing an example of the diagnosis support information for a planned passage area.

FIG. 5 is a view showing an example of the diagnosis support information for a planned passage area.

FIG. 6 is a view showing an example of the diagnosis support information for a planned passage area.

FIG. 7 is a functional block diagram of an area determination portion.

FIG. 8 is a diagram showing an inference model of a diagnosis support information generation portion.

FIG. 9 is an example of diagnosis support information that takes into account individual differences in a lumen of a hollow organ for each class of a patient.

FIG. 10 is an example of diagnosis support information that takes into account individual differences in a lumen of a hollow organ for each class of a patient.

FIG. 11 is a diagram illustrating training data used in training the inference model.

FIG. 12 is a diagram showing an example of a composite image generated by a display control portion.

FIG. 13 is a diagram showing an example of the composite image generated by the display control portion.

FIG. 14 is a control flowchart of the endoscope system.

### DETAILED DESCRIPTION

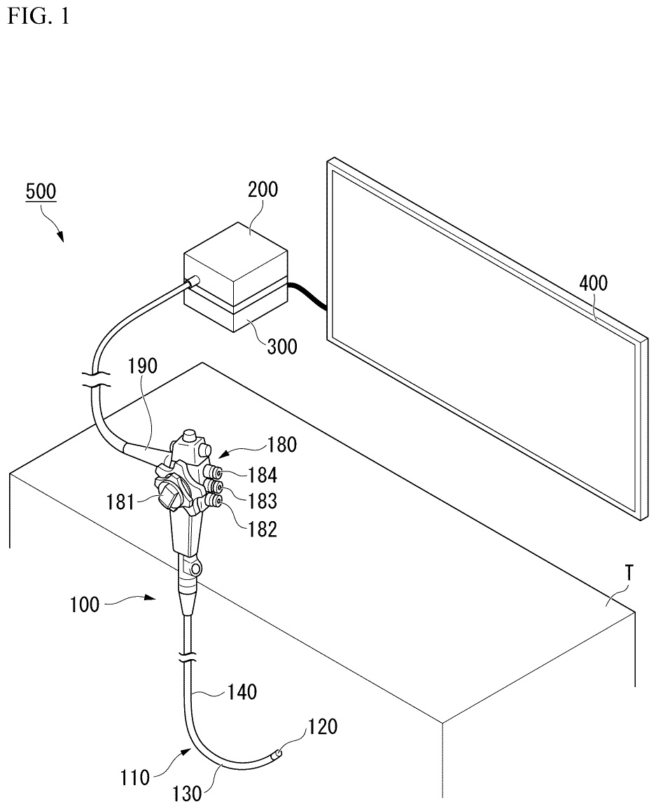

An endoscope system 500 according to one embodiment of the present disclosure will be described with reference to FIGS. 1 to 14.

### [Endoscope System 500]

FIG. 1 is a view showing the endoscope system 500.

The endoscope system 500 includes an endoscope 100, an image processing processor device 200, a light source device 300, and a display device 400. The image processing processor device 200 and the light source device 300 may be an integrated device (image control device).

The light source device 300 has a light source 310 such as an LED and controls the light source to control the amount of illumination light to be transmitted to the endoscope 100 via a light guide 161.

The display device 400 is a device that displays images generated by the image processing processor device 200, various items of information related to the endoscope system 500, and the like. The display device 400 is, for example, a liquid crystal monitor or a head-mounted display.

### [Endoscope 100]

The endoscope 100 is a device for observing and treating the inside of the body of a patient lying on a surgical table T, for example. The endoscope 100 includes an elongated insertion section 110 that is inserted into the body of the patient, an operation portion 180 that is connected to a proximal end of the insertion section 110, and a universal cord 190 that extends from the operation portions 180.

The insertion section 110 has a distal end portion 120, a bendable bending portion 130, and a long and flexible tube portion 140. The distal end portion 120, the bending portion 130, and the flexible tube portion 140 are connected in that order from a distal end side. The flexible tube portion 140 is connected to the operation portion 180.

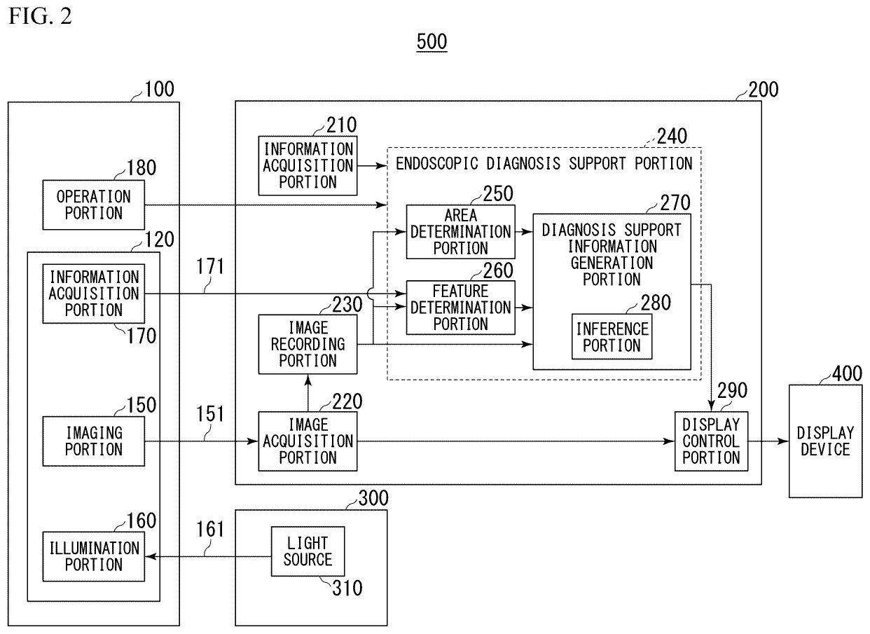

FIG. 2 is a functional block diagram of the endoscope system 500.

The distal end portion 120 has an imaging portion 150, an illumination portion 160, and an information acquisition portion 170.

The imaging portion 150 has an optical system, an imaging element that converts an optical signal into an electrical signal, and an AD conversion circuit that converts an analog signal output by the imaging element into a digital signal. The imaging portion 150 captures an image of a subject and generates an image signal. The image signal is acquired by the image processing processor device 200 via an image signal cable 151.

The illumination portion 160 irradiates the subject with illumination light transmitted by the light guide 161. The light guide 161 is inserted through the insertion section 110, the operation portion 180, and the universal cord 190 and is connected to the light source device 300. The illumination portion 160 may include a light source such as an LED, an optical element such as a fluorescent body having a wavelength conversion function, or the like.

The information acquisition portion 170 detects the position of the distal end portion 120 and the speed and direction of the distal end portion 120. The information acquisition portion 170 is, for example, a six-axis sensor or a three-axis sensor. The output of the information acquisition portion 170 is acquired by the image processing processor device 200 via a signal cable 171.

The information acquisition portion 170 detects the state, the position, or the like of the distal end portion 120 and can detect a direction of gravity from an acceleration, and the process of movement of the distal end portion 120 from a change in this direction. The information acquisition portion 170 may be any unit that can achieve the same purpose, may be a magnetic sensor or a member that generates magnetism, or may be one that detects movement or magnetism in cooperation with an external sensor or system. In addition, since the movement of the distal end portion 120 can be detected from the change in the image output acquired by the imaging portion 150, the imaging portion 150 may be used in place of the information acquisition portion 170. In addition, the information acquisition portion 170 may also be realized in cooperation with the above-described means.

The operation portion 180 receives operations on the endoscope 100. The operation portion 180 has an ankle knob 181 for controlling the bending portion 130, an air/water supply button 182, a suction button 183, and a release button 184. The operations input to the air/water supply button 182, the suction button 183, and the release button 184 are acquired by the image processing processor device 200. The release button 184 is a push button through which an operation to save a captured image acquired from the imaging portion 150 is input.

The universal cord 190 connects the endoscope 100 and the image processing processor device 200. The universal cord 190 is a cable through which the image signal cable 151, the light guide 161, the signal cable 171, and the like are inserted.

### [Image Processing Processor Device 200]

As shown in FIG. 2, the image processing processor device 200 includes an information acquisition portion 210, an image acquisition portion 220, an image recording portion 230, an endoscopic diagnosis support portion 240, and a display control portion 290.

The image processing processor device 200 is a computer capable of executing a program and equipped with a processor such as a CPU, a memory, a recording portion, and the like. The functions of the image processing processor device 200 are realized by the processor executing a program. At least some of the functions of the image processing processor device 200 may be realized by a dedicated logic circuit mounted in an ASIC or FPGA.

The image processing processor device 200 may further include components other than the processor, the memory, and the recording portion. For example, the image processing processor device 200 may further include an image computing portion that performs some or all of the image processing and image recognition processing. By further including the image computing portion, the image processing processor device 200 can execute specific image processing and image recognition processing at high speed. The image computing portion may be a computing portion provided in a cloud server connected via the Internet.

The recording portion is a non-volatile recording medium that stores the above-described program and data required for executing the program. The recording portion includes, for example, a writable non-volatile memory such as a flexible disk, a magneto-optical disk, a ROM, or a flash memory, a portable medium such as a CD-ROM, a storage device such as a hard disk or a SSD built into a computer system, or the like. The recording portion may be a storage device or the like provided in a cloud server connected via the Internet.

The above program may be provided by a “computer-readable recording medium” such as a flash memory. The program may be transmitted from a computer that holds the program to the memory or the recording portion via a transmission medium or by a transmission wave in the transmission medium. The “transmission medium” that transmits the program is a medium that has a function of transmitting information. The medium that has a function of transmitting information includes a network (communication network) such as the Internet and a communication channel (communication line) such as a telephone channel. The above-described program may realize some of the above-described functions. Furthermore, the above-described program may be a differential file (differential program). The above-described functions may be realized by a combination of a program already recorded in the computer and a differential program.

The information acquisition portion 210 controls the entire image processing processor device 200. In addition, the information acquisition portion 210 acquires information related to a case of a disease in which the endoscope system 500 is used (type of the endoscope 100, patient information, surgeon information) from an in-hospital system or the like. In addition, the information acquisition portion 210 may acquire information related to the case of a disease by having a surgeon or assistant input information from an input device (not shown). The information input to the information acquisition portion 210 is acquired by the endoscopic diagnosis support portion 240.

The image acquisition portion 220 acquires an image signal from the imaging portion 150 of the endoscope 100 via the image signal cable 151. The image acquisition portion 220 performs image signal processing on the image signals acquired from the imaging portion 150 to sequentially acquire captured images D. The image acquisition portion 220 outputs the acquired captured images D to the display control portion 290. In addition, the image acquisition portion 220 outputs the acquired captured images D to the endoscopic diagnosis support portion 240 via the image recording portion 230.

The image recording portion 230 is a part of the recording portion described above and is a non-volatile recording medium. The image recording portion 230 may be a part of the memory described above and may be a volatile recording medium. The image recording portion 230 records a plurality of captured images D that have been transferred.

The image recording portion 230 records the plurality of captured images D (image frames, time-series images) input in chronological order. When the recording capacity of the image recording portion 230 is insufficient, the oldest captured image D is deleted. The plurality of captured images D recorded in the image recording portion 230 may be captured images D of consecutive frames or may be captured images D in which a plurality of frames are thinned out from consecutive frames. The image recording portion 230 temporarily records each frame obtained from the imaging portion 150 and compares the previous and next frames, thereby making it possible to acquire information similar to that acquired by the information acquisition portion 170. By comparing the images of the previous and next frames, in a case in which the image spreads from the center of a screen to the periphery, it can be determined that the distal end portion 120 is being inserted along a lumen of a hollow organ, and in a case in which there is a change such that the image of the periphery moves toward the center of a screen, it can be determined that the distal end portion 120 is being withdrawn.

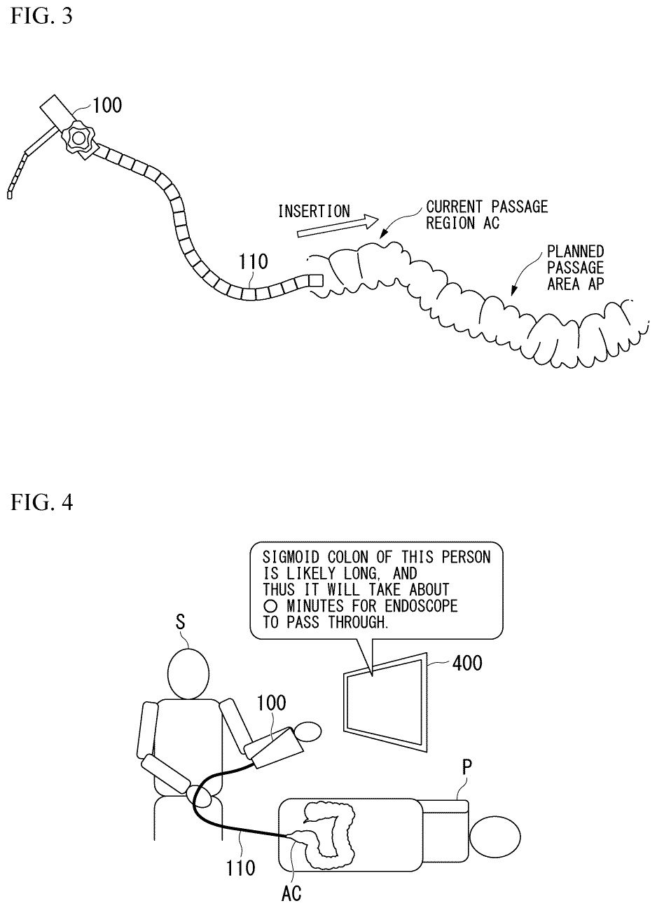

FIG. 3 is a view illustrating diagnosis support information.

For example, as shown in FIG. 3, the large intestine is made up of a plurality of connected cylindrical tubes, and there are individual differences in shape and size for each patient (subject) P. For this reason, the time required to insert the endoscope 100 forward in an advancing direction and withdraw the endoscope 100 while bending the insertion section 110 of the endoscope 100 along the series of cylindrical tubes varies depending on the individual differences in the large intestine for each patient P and the skill of a surgeon S. On the other hand, it is difficult to extend the examination time or the treatment time due to factors such as the time required for medical checkup and the effective duration of anesthesia. Therefore, in order to support the surgeon S, the endoscopic diagnosis support portion 240 provides diagnosis support for a planned passage area AP taking into account the individual differences in the large intestine for each patient P. In addition, it is desirable that the content and level of detail of the diagnosis support for the planned passage area AP be changed according to the skill of the surgeon S.

The endoscopic diagnosis support portion 240 can also provide diagnosis support for the planned passage area AP in a lumen of a hollow organ other than the large intestine. For example, in order to support the surgeon S, the endoscopic diagnosis support portion 240 provides diagnosis support for a planned passage area AP taking into account the individual differences in a stomach for each patient P.

Specifically, the endoscopic diagnosis support portion 240 determines the features of a current passage area AC of the lumen imaged in the captured image D. Next, the endoscopic diagnosis support portion 240 generates diagnosis support information for the planned passage area AP, taking into account the individual differences in the lumen for each patient P, based on the features of the current passage area AC. Here, since there is a correlation between the areas of the lumen, it is possible to predict the features of the planned passage area AP, taking into account the individual differences in the lumen for each patient P, from the features of the current passage area AC.

The endoscopic diagnosis support portion 240 generates diagnosis support information for the planned passage area AP, taking into account the patient information (the gender, the age, the race, the body type, the medical checkup results, and the like) acquired by the information acquisition portion 210 as necessary. For example, it has been reported that “the inner diameter of the large intestine is positively correlated with the height and the weight and is negatively correlated with the age for both the transverse and sigmoid colons,” that “transverse colon prolapse is correlated with the gender, the age, the height, the weight, the obesity level, and the length of the transverse colon and large intestine,” and that “the sigmoid colon elevation is correlated with the age and the length of the sigmoid colon and large intestine.” For this reason, by taking into account the patient information acquired by the information acquisition portion 210, the endoscopic diagnosis support portion 240 can concretize the correlation in the areas of the lumen for each class (attribute) of the patient P and can more accurately predict the features of the diagnosis support information for the planned passage area AP.

The planned passage area AP is an area located in front of the current passage area AC in the advancing direction of the endoscope 100. As shown in FIG. 3, when the insertion section 110 of the endoscope 100 is inserted into the lumen, the planned passage area AP is an area in the lumen which is deeper than the current passage area AC. When the insertion section 110 of the endoscope 100 is withdrawn from the lumen, the planned passage area AP is an area in the lumen which is closer to the natural opening than the current passage area AC.

FIG. 4 is a view showing an example of the diagnosis support information for the planned passage area AP. As shown in FIG. 4, in a case in which the surgeon S inserts the insertion section 110 of the endoscope 100 into the large intestine, when the current passage area AC is a “rectum,” the endoscopic diagnosis support portion 240 generates diagnosis support information for the “sigmoid colon,” which is the planned passage area AP, for example. The diagnosis support information illustrated in FIG. 4 is a predicted time required for the surgeon S to examine the planned passage area AP.

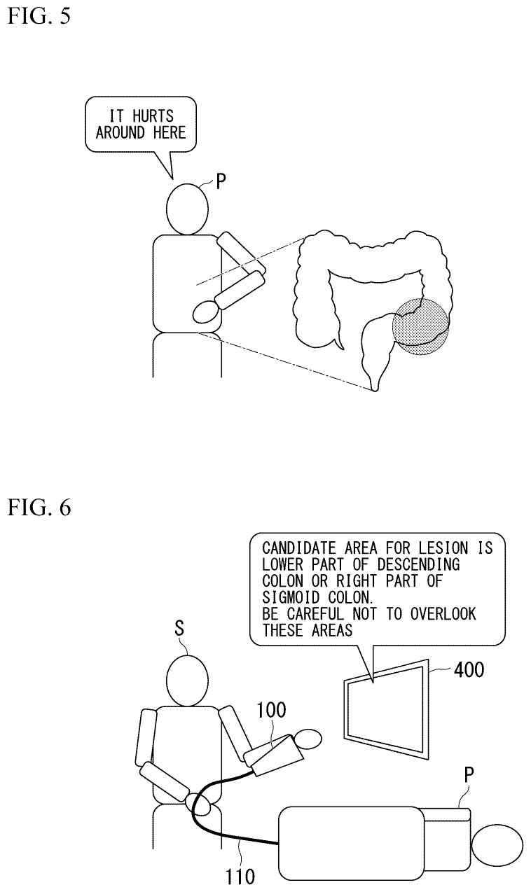

FIGS. 5 and 6 are views showing an example of the diagnosis support information for the planned passage area AP.

As shown in FIG. 5, in a case in which the medical checkup results, such as a portion where the patient P is in pain, can be acquired in advance, the endoscopic diagnosis support portion 240 generates diagnosis support information for a candidate area where a lesion is located (also referred to as a “specific target area, specific part”). The diagnosis support information illustrated in FIG. 6 notifies the surgeon S that the candidate area for the lesion is a lower part of the descending colon or a right part of the sigmoid colon and urges the surgeon S to observe carefully.

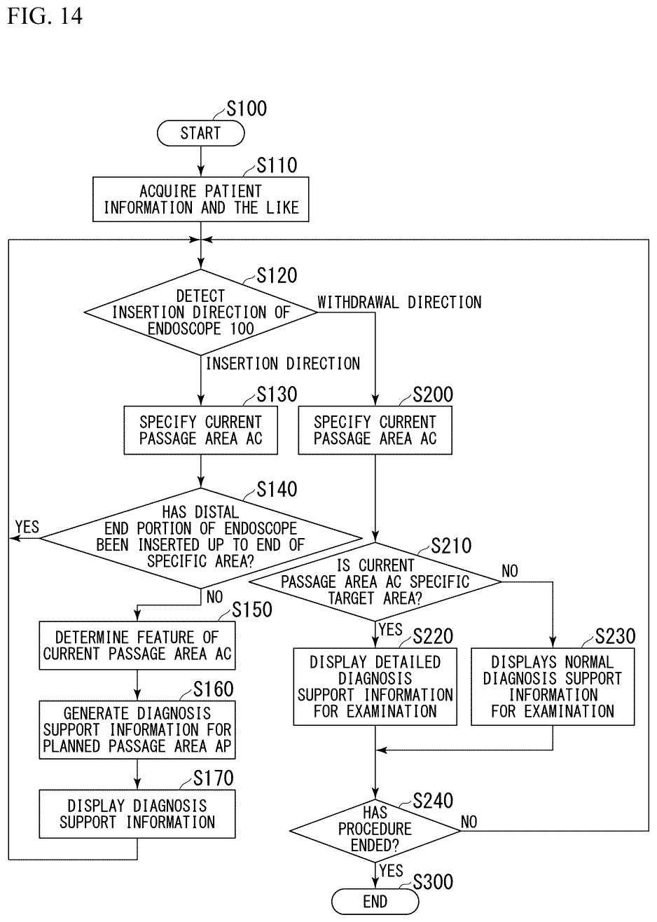

A main symptom among symptoms that a patient mentions to a doctor is called a chief complaint. In the case of the example shown in FIG. 5, the chief complaint is a symptom of “it hurts here” while the patient points to his side. The endoscopic diagnosis support portion 240 can provide diagnosis support information that enables an appropriate diagnosis for such a chief complaint. For example, the patient may explain other concerns, but in this case, the patient clearly complains of abdominal pain, which may be related to the endoscopic examination, and thus which will be the chief complaint related to this examination. By simply touching this area with a hand of the patient or subject, the doctor can determine anatomically where this area is located within a digestive organ lumen in the endoscopic examination. The doctor may input information obtained from such a chief complaint (also referred to as “chief complaint information”) as some of the patient information (S110 in FIG. 14, which will be described below).

In addition, the endoscopic diagnosis support portion 240 may acquire the chief complaint information by reading the results of the patient writing a check mark on a sheet of paper on which a shape that resembles a human body is represented, or may acquire the chief complaint information by displaying a UI display of a shape that resembles a human body on a terminal and reading a check mark input to the UI display. The endoscopic diagnosis support portion 240 may be provided with a program or database that determines more specifically which part of which organ the part mentioned by the patient is, based on the input results.

As shown in FIG. 2, the endoscopic diagnosis support portion 240 includes an area determination portion 250, a feature determination portion 260, and a diagnosis support information generation portion 270.

The endoscopic diagnosis support portion 240 may be a device (hereinafter also referred to as an “endoscopic diagnosis support device”) separated from the image processing processor device 200. The endoscopic diagnosis support device may be a computing device provided in a cloud server connected via the Internet. The computing device provided on the cloud server may have an image acquisition portion and may be equipped with a determination function that determines the position of the endoscope distal end portion from the acquired image, and a determination function that determines the features of the area through which the distal end portion 120 of the endoscope is currently passing (features of the current passage area of the distal end portion) from information acquired by the distal end portion 120. Depending on the system, the image recording portion may be provided in the computing device. In addition, although the image is acquired via the image recording portion 230 in FIG. 2, there are cases in which it is not necessary to go through the image recording portion 230. The area determination portion 250 and the feature determination portion 260 acquire intraluminal feature information obtained in the process of inserting the endoscope into the digestive organ lumen in the endoscopic examination and thus may be called an intraluminal feature information acquisition portion.

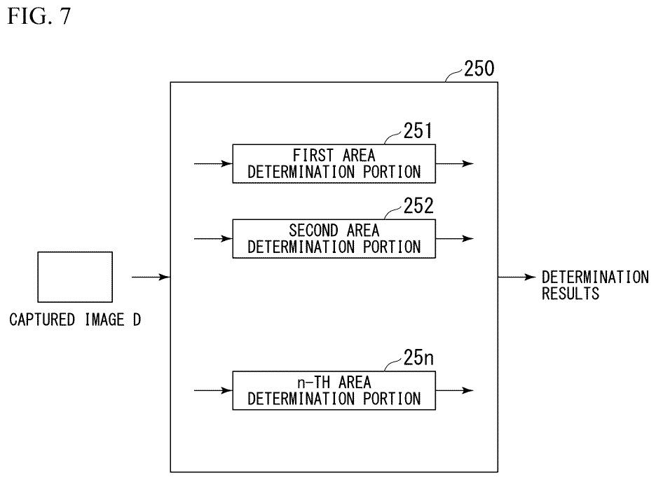

FIG. 7 is a functional block diagram of the area determination portion 250.

The area determination portion 250 specifies the current passage area AC of the lumen imaged in the captured image D. When the insertion section 110 of the endoscope 100 is inserted into the large intestine, the area determination portion 250 analyzes the obtained image and specifies whether the area of the large intestine included in the captured image D is one of the cecum, the ascending colon, the transverse colon, the descending colon, the sigmoid colon, the rectosigmoid portion, and the like.

The area determination portion 250 illustrated in FIG. 7 has a plurality of area determination portions (a first area determination portion 251, a second area determination portion 252, and an n-th area determination portion 25n). Each area determination portion is a dedicated determination portion for each area of the lumen. For example, in a case in which the lumen is the large intestine, the first area determination portion 251 is a determination portion that determines that the current passage area AC of the lumen imaged in the captured image D is the “rectum.” In addition, the second area determination portion 252 is a determination portion that determines that the current passage area AC of the lumen imaged in the captured image D is the “sigmoid colon.”

Each area determination portion may specify the current passage area AC of the lumen included in the captured image D by pattern matching. For example, each area determination portion compares pre-recorded images of each part with the captured image D and specifies the current passage area AC of the lumen included in the captured image D based on the similarity to the pre-recorded images of each part.

Each area determination portion may specify the current passage area AC of the lumen included in the captured image D using a machine learning model. For example, each area determination portion specifies the current passage area AC of the lumen included in the captured image D using a machine learning model that has been trained in advance to be able to detect the area of the lumen included in the captured image D from the captured image D.

Each area determination portion may specify the current passage area AC of the lumen included in the captured image D based on the output of the information acquisition portion 170 (the speed, the direction, the posture, and the like of the distal end portion 120).

The region determination portion 250 may specify the current passage area AC of the lumen imaged in a plurality of captured images D using only an area determination portion that can distinguish and specify a plurality of areas of the lumen without using a plurality of area determination portions.

The area determination portion 250 may specify the current passage area AC of the lumen included in the captured image D by combining the above-described plurality of techniques.

The area determination portion 250 transmits the specified area of the lumen included in the captured image D to the diagnosis support information generation portion 270.

The feature determination portion 260 determines the features of the current passage area AC from the obtained image signal. The features of the current passage area AC determined by the feature determination portion 260 include, for example, the shape (the intraluminal length, the inner diameter, or the like) and the surface condition (the wall condition, the blood vessel condition, or the like). The intraluminal length is the length in a direction of extension of the lumen.

The feature determination portion 260 may determine the features of the current passage area AC by performing image processing on the captured image D.

The feature determination portion 260 may determine the features of the current passage area AC based on the output of the information acquisition portion 170 (the speed, the direction, the posture, and the like of the distal end portion 120). For example, the feature determination portion 260 can determine the intraluminal length of the current passage area AC from the speed and direction of the distal end portion 120.

The feature determination portion 260 may determine the features of the current passage area AC based on the operation history input to the operation portion 180. For example, in a case in which a plurality of operations of supplying water to the current passage area AC have been performed, the feature determination portion 260 can determine that the current passage area AC is one of the areas where residue is likely to accumulate.

The feature determination portion 260 may determine the features of the current passage area AC by combining the above-described plurality of techniques.

The feature determination portion 260 transmits the determined area of the lumen included in the captured image D to the diagnosis support information generation portion 270.

The diagnosis support information generation portion 270 generates diagnosis support information for the planned passage area AP based on the captured image D and the specification results and features of the current passage area AC. The diagnosis support information generation portion 270 may generate the diagnosis support information on a rule basis or may generate the diagnosis support information using an inference model 281 included in an inference portion 280. The diagnosis support information generation portion 270 can also be described as one that outputs guide information when accessing a specific part prior to diagnosis and can therefore be rephrased as a guide information output portion that outputs guide information when the distal end portion 120 of the endoscope 100 is inserted further from a passing area in accordance with the intraluminal feature information to access a specific target part.

The inference portion 280 may use conventional general-purpose arithmetic processing circuits such as a CPU or a field programmable gate array (FPGA), but much of the processing in neural networks involves matrix multiplication, and thus it may also use a graphic processing unit (GPU) or a tensor processing unit (TPU) that is specialized for matrix calculations. In recent years, such artificial intelligence (AI) dedicated hardware, called “neural network processing units (NPUs),” have been designed to be integrated and embedded with other circuits such as CPUs and may even become part of the processing circuits.

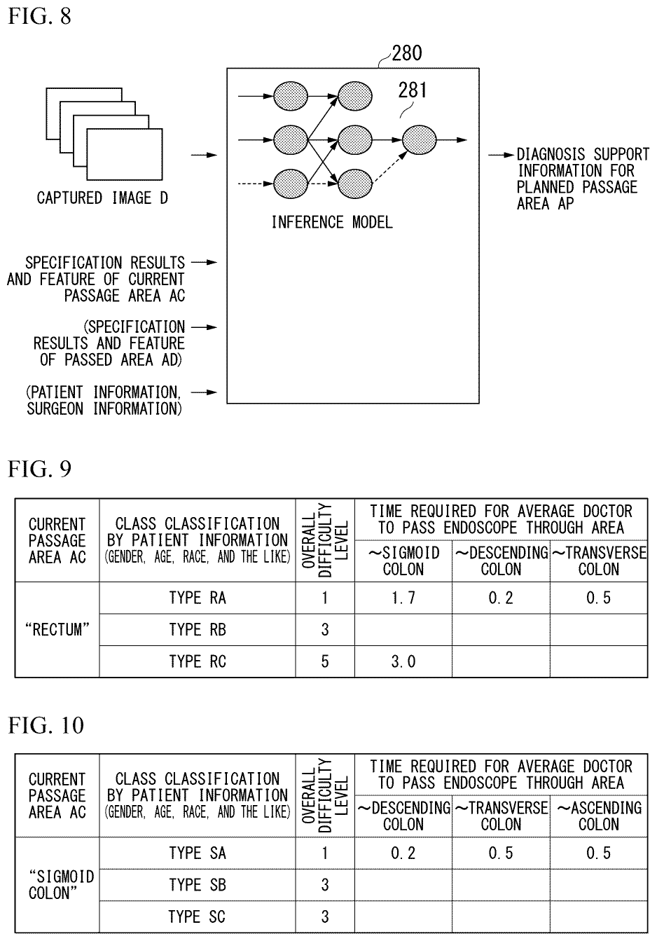

FIG. 8 is a diagram showing the inference model 281 and an image of how it is trained.

The inference model 281 is a model that has been trained using training data including annotations related to diagnosis support information for the planned passage area AP for a plurality of image frames (captured images for training) in a plurality of cases of diseases. The inference model 281 is, for example, a neural network, and is trained using deep learning. The inference model 281 is not limited to a neural network but may be any other machine learning model that can output information for an input image.

The input to the inference model 281 includes the plurality of captured images D (image frames, time-series images) input in chronological order, and the specification results and features of the current passage area AC. The output of the inference model 281 includes the diagnosis support information for the planned passage area AP.

The input to the inference model 281 may include patient information and surgeon information acquired by the information acquisition portion 210. For example, by inputting the patient information of the patient P (the gender, the age, the race, the body type, the medical checkup results, and the like) into the inference model 281, the inference model 281 can more easily output the diagnosis support information that takes into account the individual differences in the lumen for each class (attribute) of the patient P. For example, by inputting the skill (proficiency) of the surgeon S, which is the surgeon information, into the inference model 281, the inference model 281 can more easily output the diagnosis support information that corresponds to the skill of the surgeon S.

In this way, by training the inference model 281 using the training data in which an image of a first section obtained in the process of inserting of the endoscope into the digestive organ lumen in the endoscopic examination is annotated with feature information of a second section into which the endoscope is inserted at a later timing than the first section, it is possible to provide an endoscope guide control method that includes the display control portion 290 that infers and displays the feature information of the second section which is inferred by the inference model 281 when an image corresponding to the first section of the intraluminal image acquired from the imaging portion 150 of the endoscope 100 is input to the inference model 281. In addition, when the diagnosis support information generation portion 270 is referred to as a “guide information output portion,” it can also be expressed as one that is equipped with an inference model that has been trained using the training data in which the image of the first section obtained in the process of the passage of the distal end portion of the endoscope is annotated with the feature information of the second section that the distal end portion 120 of the endoscope 100 reaches after being inserted further from the passing area. When inserting the endoscope 100, it is impossible to know what will be at the end of the insertion, and thus it is important to have a mechanism to predict this. Therefore, although it has been described that the first section is closer to the insertion position and the second section is further deep, the opposite is also possible. This concept can also be used when removing the endoscope 100. This is because the lumen is soft and may assume different shapes during insertion and withdrawal.

In addition, in a case in which a subject undergoing an endoscopic examination has the chief complaint information such as a concern, there is a need to closely observe that part. Therefore, the doctor can prepare himself/herself mentally by receiving some kind of guidance before the imaging portion 150 of the distal end portion 120 of the endoscope 100 moves to the specific part at a timing prior to the observation of the part. In other words, if the endoscopic image obtained in the process of inserting of the endoscope into the digestive organ lumen in the digestive organ endoscopic examination of the subject are acquired, and if a specific part is determined based on the chief complaint information, it is possible to obtain the inference model 281 that has been trained using the training data in which the endoscopic image of a section which is acquired at a timing prior to the specific part for the endoscopic image in the inserting process is annotated with information related to the specific part. Therefore, by also using the endoscope guide control method that includes a display control step of inferring and displaying information regarding the specific part which is inferred by the inference model 281 when the intraluminal image acquired from the imaging portion 150 of the endoscope 100 is input, it is possible to perform an accurate medical checkup without overlooking any points of concern to the patient.

FIG. 9 is an example of the diagnosis support information that takes into account the individual differences in the lumen for each class of the patient P. In a case in which the surgeon S inserts the insertion section 110 of the endoscope 100 into the large intestine, when the current passage area AC is the “rectum,” the diagnosis support information for the “sigmoid colon,” the “descending colon,” and the “transverse colon,” which are the planned passage areas AP for each class of the patient P is output. The diagnosis support information illustrated in FIG. 9 is a predicted time required for an average surgeon to examine (pass the endoscope through) the planned passage area AP. The predicted time required for the examination is calculated based on, for example, the intraluminal length (estimated value) of the planned passage area AP. The diagnosis support information may be the difficulty level for each planned passage area AP.

FIG. 10 is an example of the diagnosis support information that takes into account the individual differences in the lumen for each class of a patient P. In a case in which the surgeon S inserts the insertion section 110 of the endoscope 100 into the large intestine, when the current passage area AC is the “sigmoid colon,” the diagnosis support information for the “descending colon,” the “transverse colon,” and the “ascending colon,” which are the planned passage areas AP for each class of the patient P is output. The diagnosis support information illustrated in FIG. 10 is a predicted time required for an average surgeon to examine (pass the endoscope through) the planned passage area AP. The predicted time required for the examination is calculated based on, for example, the intraluminal length (estimated value) of the planned passage area AP. The diagnosis support information may be the difficulty level for each planned passage area AP.

The input of the inference model 281 may include the specification results and the features of the passed area AD. The passed area AD is an area which is located behind the current passage area AC in the advancing direction of the endoscope 100 and is an area through which the distal end portion 120 of the endoscope 100 has already passed. For example, in a case in which the surgeon S inserts the insertion section 110 of the endoscope 100 into the large intestine, when the current passage area AC is the “sigmoid colon,” one of the passed areas AD is the “rectum.” By inputting the specification results and features of the passed area AD into the inference model 281, the inference model 281 can more accurately output the diagnosis support information for the planned passage area AP. As the examination and diagnosis progress, the passed area AD increases, and therefore the diagnosis support information for the planned passage area AP becomes more accurate.

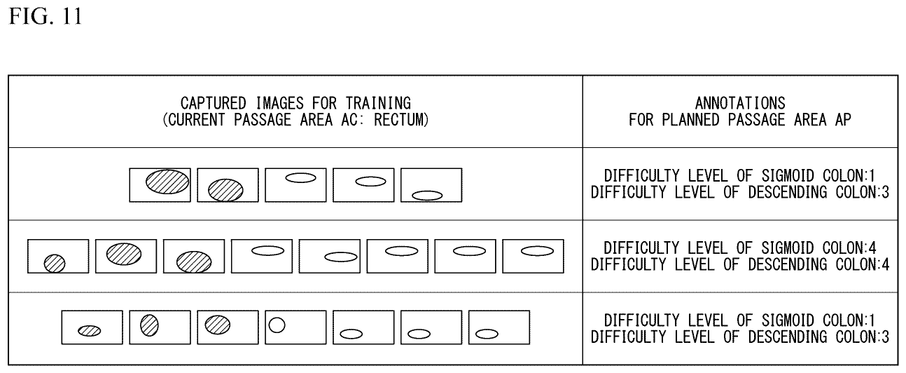

FIG. 11 is a diagram illustrating the training data.

As the training data, a plurality of image frames (a series of still images) obtained during endoscopic examinations in a plurality of cases of diseases used. The training data is a combination of a plurality of image frames (captured images for training) and annotations related to diagnosis support information for the planned passage area AP. The inference model 281 is a model that has been trained using training data such that a corresponding annotation is output for an input image frame (captured image for training).

The annotations of the training data may include measures for retrieving. The measures for retrieving might be, for example, “the difficulty level of the planned passage area AP is extremely high, and thus support of veterans or experts should be required.” In a case in which the annotations of the training data include the measures for retrieving, the inference model 281 can further output the measures for retrieving.

The diagnosis support information generation portion 270 may generate diagnosis support information for the planned passage area AP based on the captured image D only. In this case, the inference model 281 is a model that outputs diagnosis support information for the planned passage area AP from only the current passage area AC of the lumen imaged in the captured image D.

The diagnosis support information generation portion 270 may generate diagnosis support information for the planned passage area AP based on the specification results and features of the current passage area AC only.

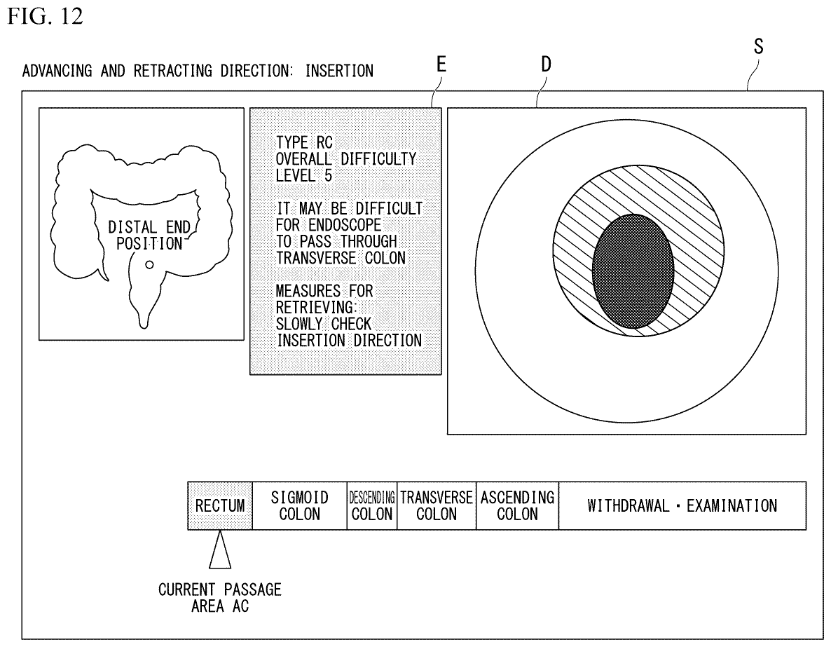

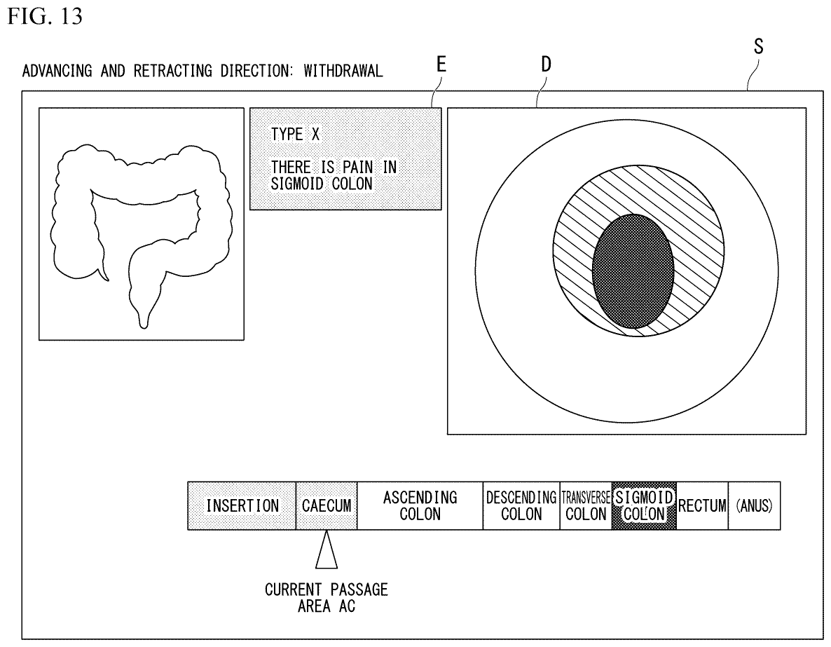

FIGS. 12 and 13 are diagrams each showing an example of a composite image S.

The display control portion (image compositing portion) 290 generates the composite image S including the captured image D and diagnosis support information E.

The composite image S shown in FIG. 12 is generated when the surgeon S inserts the insertion section 110 of the endoscope 100 into the large intestine (advancing and retracting direction: insertion). The diagnosis support information E illustrated in FIG. 12 includes the class (attribute) of the patient P, the overall difficulty level of the planned passage area AP, the presentation of the “transverse colon” which is an area having a particularly high difficulty level, and the measures for retrieving.

The composite image S shown in FIG. 13 is generated when the surgeon S is performing an examination while withdrawing the insertion section 110 of the endoscope 100 from the large intestine (advancing and retracting direction: withdrawal). The diagnosis support information E illustrated in FIG. 13 includes the class (attribute) of the patient P and the presentation of the specific target area (for example, an area corresponding to a portion where the patient P complains of pain and which is a candidate area for a lesion).

### [Operation of Endoscope System 500]

Next, the operation of the endoscope system 500 (diagnosis support method) will be described. Specifically, a procedure for observing and treating a lumen wall of a hollow organ in the large intestine using the endoscope system 500 will be described. The endoscope system 500 can also perform diagnosis support for organs other than the large intestine (such as the stomach, the bronchi, and the urinary organs). Hereinafter, a description will be given along a control flowchart of the endoscope system 500 shown in FIG. 14.

### <Step S110>

In step S110, the information acquisition portion 210 acquires information related to the case of a disease (type of the endoscope 100, patient information, and surgeon information). The chief complaint information may be input in step S110. The information acquisition portion 210 may determine, from the input chief complaint information, which part should be observed closely in the upcoming examination, by referring to a specific program or database. The information acquisition portion 210 may acquire information related to the condition of the patient by having a surgeon or assistant input information from an input device (not shown). The endoscope system 500 then executes step S120.

### <Step S120>

In step S120, the area determination portion 250 detects the insertion direction of the endoscope 100. Specifically, the area determination portion 250 acquires the output of the information acquisition section 170 and detects the advancing and retracting direction (insertion direction, withdrawal direction) of the distal end portion 120 of the endoscope 100 based on the output of the information acquisition portion 170. The area determination portion 250 may specify the area of the lumen imaged in the captured image D from the captured image D and detect the advancing and retracting direction of the endoscope 100 from the history of the specified region. In a case in which the insertion direction of the endoscope 100 is the insertion direction, the endoscope system 500 then executes step S130. In a case in which the insertion direction of the endoscope is the withdrawal direction, the endoscope system 500 then executes step S200.

### <Step S130>

In step S130, the area determination portion 250 specifies the current passage area AC of the lumen imaged in the captured image D. Then, the endoscope system 500 executes step S140.

### <Step S140>

In step S140, the area determination portion 250 determines whether the distal end portion 120 of the endoscope 100 has been inserted up to the end of the specific area such as the cecum. In a case in which the distal end portion 120 of the endoscope 100 has been inserted up to the end of the specific area, the endoscope system 500 executes step S120. In a case in which the distal end portion 120 of the endoscope 100 has not been inserted up to the end of the specific area, the endoscope system 500 executes step S150.

### <Step S150>

In step S150, the feature determination portion 260 determines the features of the current passage area AC. Based on the image information or the like obtained at this time, image data information for outputting information on the part to be examined at a later timing is acquired by the inference model 281, other databases, or the like. Then, the endoscope system 500 executes step S160.

### <Step S160>

In step S160, the diagnosis support information generation portion 270 generates diagnosis support information for the planned passage area AP based on the captured image D and the specification results and features of the current passage area AC. The diagnosis support information which is inferred by the inference model 281 when the intraluminal image acquired from the imaging portion 150 of the endoscope 100 is input may be inferred (The diagnosis support information does not have to be the diagnosis itself but includes various information to enable diagnosis. The diagnosis support information may be auxiliary information for access. Alternatively, the diagnosis support information may include information on various operations such as water injection, air supply, and suction, or switching of light sources, switching of image processing, spraying of reagents, and the like). Due to this, the diagnosis support information becomes guide information that predicts what will happen next when the intraluminal image acquired from the imaging portion 150 of the endoscope 100 is input. For example, it is possible to provide an endoscope guide control method that uses the inference model 281 and includes a display control step of inferring and displaying information on the specific area (information related to ease of access and the relationship to the chief complaint of the patient, as well as observation and diagnostic know-how) inferred when the endoscopic image is input into the inference model 281. The information on the specific part in the digestive organ lumen here may be determined from the information obtained from the chief complaint of the subject, but as shown in FIG. 8 as “the specification results of the current passage area AC,” may be determined according to the information on the part currently being observed (passing through) in this digestive organ lumen examination. Then, the endoscope system 500 executes step S170.

### <Step S170>

In step S170, the display control portion 290 generates the composite image S including the captured image D and the diagnosis support information E and outputs the composite image S to the display device 400. The display device 400 displays the composite image S. Then, the endoscope system 500 executes step S120. Because the doctor can grasp the information before reaching the examination portion, they can be mentally prepared and access and examine the portion accurately and precisely without having to hesitate to make a decision in the event of an unexpected situation where insertion becomes difficult or overlooking any points of concern to the patient.

The display control portion 290 may present and notify the user of the diagnosis support information according to the content of the generated diagnosis support information at an early stage. For example, in a case in which the difficulty level of the planned passage area AP is extremely high in consideration of the skill of the surgeon S, the diagnosis support information for the surgeon S is presented and notified to the user at an early stage.

### <Step S200>

In step S200, the area determination portion 250 specifies the current passage area AC of the lumen imaged in the captured image D. Then, the endoscope system 500 executes step S210.

### <Step S210>

In step S210, the area determination portion 250 determines whether the current passage area AC is the specific target area (for example, an area corresponding to a portion where the patient P complains of pain and which is a candidate area for a lesion). In a case in which the current passage area AC is the specific target area, the endoscope system 500 then executes step S220. In a case in which the current passage area AC is not the specific target area, the endoscope system 500 then executes step S230.

### <Step S220>

In step S220, the diagnosis support information generation portion 270 displays detailed diagnosis support information for the examination. Specifically, as shown in FIG. 6, the surgeon S is notified that the current passage area AC or the planned passage area AP is a candidate area for a lesion. Furthermore, the diagnosis support information generation portion 270 executes the same processing as in steps S150 to S170, generates the composite image S including the captured image D and the diagnosis support information E and outputs the composite image S to the display device 400. Then, the endoscope system 500 executes step S240.

### <Step S230>

In step S230, the diagnosis support information generation portion 270 displays normal diagnosis support information for the examination. Specifically, the diagnosis support information generation portion 270 executes the same processing as in steps S150 to S170, generates the composite image S including the captured image D and the diagnosis support information E and outputs the composite image S to the display device 400. Then, the endoscope system 500 executes step S240.

### <Step S240>

In step S240, the endoscopic diagnosis support portion 240 determines whether the procedure has ended. In a case in which the endoscopic diagnosis support portion 240 determines that the procedure has not ended, it executes step S120 and subsequent steps. In a case in which the endoscopic diagnosis support portion 240 determines that the procedure has ended, it performs step S300 and ends the control flow shown in FIG. 14.

According to the endoscope system 500 of the present embodiment, the endoscopic diagnosis support portion 240 (endoscopic diagnosis support device) can provide diagnosis support that takes into account individual differences in a lumen of a hollow organ for each patient P without requiring any preliminary work before the surgery.

In the above, one embodiment of the present disclosure has been described in detail with reference to the drawings, but the specific configuration is not limited to the embodiment, and a design change and the like within a range not departing from the gist of the present disclosure are also included. In addition, the constituent elements shown in the above-described embodiment and a modification example shown below can be appropriately combined and configured.

In particular, the endoscope system 500 shown in FIG. 2 includes a plurality of devices, namely, the endoscope 100, the image processing processor device 200, and the light source device 300, but it may also be configured such that the image processing processor device 200 and the light source device 300 are built into the endoscope 100. In addition, the image processing processor device 200 does not necessarily need to include the information acquisition portion 210, the endoscopic diagnosis support portion 240, the display control portion 290, or the image recording portion 230, and from the standpoint of system scalability and commonality, it may work in conjunction with another processor device to achieve similar functions. In other words, the image acquisition portion that acquires an image of the lumen from the endoscope 100, the area determination portion that specifies the current passage area of the lumen, the feature determination portion that determines the features of the current passage area, and the diagnosis support information generation portion that generates the support information for passing through or diagnosing the planned passage area in the lumen based on the features of the current passage area do not necessarily need to be included within the same device, and the devices may work in conjunction with each other as appropriate to form the endoscopic diagnosis support system.

In the above embodiment, the endoscopic diagnosis support portion (endoscopic diagnosis support device) performs diagnosis support on images from a medical endoscope. However, the diagnosis target of the endoscopic diagnosis support portion (endoscopic diagnosis support device) is not limited to images from a medical endoscope. The endoscopic diagnosis support portion (endoscopic diagnosis support device) may perform diagnosis support for captured images acquired from other imaging devices such as a camera, a video camera, an industrial endoscope, a microscope, a robot with an image acquisition function, and mobile devices such as a smartphone, a mobile phone, a smartwatch, a tablet terminal, and a notebook PC.

The present disclosure can be applied to an endoscope system and the like.

What is claimed is:

1. An endoscopic diagnosis support device comprising:
an area determination portion configured to specify a current passage area of a lumen, which has been imaged in an image of the lumen of a hollow organ;
a feature determination portion configured to determine features of the current passage area; and
a diagnosis support information generation portion configured to generate support information for accessing or diagnosing a planned passage area in the lumen based on features of the current passage area.

2. The endoscopic diagnosis support device according to claim 1, wherein the features include an intraluminal length in an extension direction of the lumen.

3. The endoscopic diagnosis support device according to claim 1, wherein the diagnosis support information generation portion generates the support information based on features of a passed area that is located behind the current passage area in the lumen in the advancing direction of the endoscope.

4. The endoscopic diagnosis support device according to claim 1, wherein the diagnosis support information generation portion generates more detailed support information when the current passage area is a specific target area.

5. The endoscopic diagnosis support device according to claim 4, wherein the specific target area is an area corresponding to a portion where a patient is in pain.

6. The endoscopic diagnosis support device according to claim 2, wherein the support information is a predicted time required to examine the planned passage area calculated based on the intraluminal length of the planned passage area.

7. The endoscopic diagnosis support device according to claim 1, wherein the diagnosis support information generation portion generates the support information for the planned passage area from features of the current passage area based on an inference model that has learned a relationship between features of the current passage area and features of the planned passage area in the lumen.

8. The endoscopic diagnosis support device according to claim 7,
wherein the inference model is a model that has learned a relationship between features of a passed area that is located behind the current passage area in the lumen in an advancing direction of the endoscope and features of the planned passage area, and
wherein the diagnosis support information generation portion generates the support information for the planned passage area from features of the passed area based on the inference model.

9. The endoscopic diagnosis support device according to claim 1, wherein the diagnosis support information generation portion notifies a user of the support information according to a content of the generated support information at an early stage.

10. An endoscopic diagnosis support device comprising an inference model that has been trained using training data in which an image of a current passage area obtained in a process of inserting of an endoscope into a lumen of a hollow organ is annotated with features of a planned passage area that is located in front of the current passage area in an advancing direction of the endoscope in the lumen,
wherein the inference model infers features of the planned passage area when an image of the current passage area obtained from the endoscope is input.

11. An endoscopic diagnosis support method comprising:
specifying a current passage area of a lumen, which has been imaged in an image of the lumen of a hollow organ;
determining features of the current passage area; and
generating support information for accessing or diagnosing a planned passage area in the lumen based on features of the current passage area.

12. An endoscopic diagnosis support program causing a computer:
to specify a current passage area of a lumen, which has been imaged in an image of the lumen of a hollow organ;
to determine features of the current passage area; and
to generate support information for accessing or diagnosing a planned passage area in the lumen based on features of the current passage area.

13. A diagnosis support system comprising:
an area determination portion configured to specify a current passage area of a lumen, which has been imaged in an image of the lumen of a hollow organ;
a feature determination portion configured to determine features of the current passage area; and
a diagnosis support information generation portion configured to generate support information for passing through or diagnosing a planned passage area in the lumen based on features of the current passage area.

14. An endoscope guide control method comprising:
a step of acquiring an endoscopic image obtained in a process of insertion into or withdrawal from a lumen of a digestive organ during an endoscopic examination; and
a display control step of, with an inference model that has been trained using training data in which an endoscopic image of a first section in the process of the insertion or the withdrawal is annotated with feature information of a second section through which an endoscope passes at a later timing than the first section, inferring and displaying feature information of the second section inferred by the inference model when an image corresponding to the first section of an intraluminal image acquired from an imaging portion of the endoscope is input.

15. The endoscope guide control method according to claim 14, wherein the display control step further includes
a step of inputting an image obtained from the endoscope to an inference model that has been trained using training data in which an image of the first section is annotated with feature information of a second section that a distal end portion of the endoscope reaches while changing a position further from a passing area in the process of the insertion or the withdrawal, and
a step of adopting output results of the inference model.

16. An endoscopic examination guide device comprising:
an intraluminal feature information acquisition portion configured to acquire feature information in a lumen through which a distal end portion of an endoscope passes, which is obtained in a process of inserting an endoscope into a digestive organ lumen during an endoscopic examination; and
a guide information output portion configured to output guide information when the distal end portion of the endoscope is inserted further from a passing area in accordance with the feature information in the lumen to access a specific target part.

17. The endoscopic examination guide device according to claim 16, wherein the guide information output portion is equipped with an inference model that has been trained using training data in which an image of an first section obtained in a process of passage of the distal end portion of the endoscope is annotated with feature information of a second section that the distal end portion of the endoscope reaches after being inserted further from the passing area.

18. An endoscope guide control method comprising:
a step of acquiring information related to a specific part in a digestive organ lumen of a subject;
a step of acquiring an endoscopic image obtained in a process of insertion into a lumen of a digestive organ during a digestive organ endoscopic examination of the subject; and
a display control step of inferring and displaying information related to the specific part inferred by an inference model that has been trained using training data in which an endoscopic image of a section which is acquired at a timing prior to the specific part for the endoscopic image in the process of the insertion is annotated with the information related to the specific part when an intraluminal image acquired from an imaging portion of an endoscope is input to the inference model.

19. The endoscope guide control method according to claim 18, further comprising:
a step of inputting a chief complaint of the subject; and
a step of determining the information related to the specific part in the digestive organ lumen in accordance with chief complaint information.

20. The endoscope guide control method according to claim 18, further comprising a step of determining the information related to the specific part in the digestive organ lumen in accordance with information on a current observation part during an examination of the digestive organ lumen.
